SEQUENCE LISTING (6 sequences)

SEQ ID NO: 1 - DNA, 21 bp, Artificial
  1st pair of primers for CYP3A5 in genotyping platform example
cctgccttcaatttttcactg

SEQ ID NO: 2 - DNA, 20 bp, Artificial
  1st pair of primers for CYP3A5 in genotyping platform example
gcaatgtaggaaggagggct

SEQ ID NO: 3 - DNA, 24 bp, Artificial
  forward primer for CYP3A4 in genotyping platform example
tggcttgttgggatgaatttcaag

SEQ ID NO: 4 - DNA, 24 bp, Artificial
  reverse primer for CYP3A4 in genotyping platform example
ttactggggagtccaagggttctg

SEQ ID NO: 5 - DNA, 19 bp, Artificial
  2nd pair of primers (nested) for CYP3A5 in genotyping platform example
aatattctttttgataatg

SEQ ID NO: 6 - DNA, 22 bp, Artificial
  2nd pair of primers (nested) for CYP3A5 in genotyping platform example
cattctttcactagcactgttc

(19) United States
(12) Patent Application Publication
(10) Pub. No.: US 2006/0172334 A1
(43) Pub. Date: Aug. 3, 2006

(54) Method for detecting adverse reaction susceptibility to an HMG CoA reductase inhibitor

(75) Inventors: Russell A. Wilke, Marshfield, WI (US); James K. Burmester, Marshfield, WI (US); Richard Berg, Marshfield, WI (US)

Correspondence Address: FAEGRE & BENSON LLP;PATENT DOCKETING, 2200 WELLS FARGO CENTER, 90 SOUTH 7TH STREET, MINNEAPOLIS, MN, 55402-3901, US

(73) Assignee: Marshfield Clinic, Marshfield, WI (US)

(21) Appl. No.: 11/344,732

(22) Filed: Jan. 31, 2006

Related U.S. Application Data
(60) Provisional application No. 60/649,336, filed on Feb. 1, 2005.

(51) Int. Cl. C12Q 1/68

(52) U.S. Cl. 435/6

(57) ABSTRACT

The present invention provides a method for detecting adverse reaction susceptibility and/or severity risk to an HMG CoA reductase inhibitor by determining the genotype of a CYP3A gene of a subject.

### CROSS REFERENCE TO RELATED APPLICATIONS

This application claims the priority benefit of U.S. Provisional Application No. 60/649,336, filed Feb. 1, 2005, which is incorporated herein by reference in its entirety.

### FIELD OF THE INVENTION

The present invention relates to a method for detecting adverse reaction susceptibility and/or severity risk to an HMG CoA reductase inhibitor.

### BACKGROUND OF THE INVENTION

The association between elevated serum cholesterol and coronary artery disease is well established. In recent years, the HMG CoA reductase inhibitors (e.g., “statins”) have become the method of choice in cholesterol-lowering therapy. Currently, atorvastatin (Lipitor) is the most commonly prescribed agent within this class of drugs. See, for example, Jones et al., Am. J. Cardiol., 2003, 92, 152-160. Although statins in general are regarded as safe, one potential adverse effect has become an area of clinical concern. Approximately 1-5% of patients using this class of drugs develop muscle complaints. These complaints range from mild discomfort (myalgia) to muscle damage (myopathy). Thompson et al., JAMA, 2003, 289, 1681-1690; and Newman et al., Am. J. Cardiol., 2003, 92, 670-676. When statins are prescribed as monotherapy, the incidence of severe myopathy is relatively low (less than 1%). However, cases of severe myopathy (rhabdomyolysis) have been reported when atorvastatin was prescribed along with other drugs.

Atorvastatin-induced muscle damage (myopathy) may be accompanied by leakage of muscle enzymes into the blood, and this process can be monitored clinically by measuring circulating levels of creatine kinase (CK). However, the CK level is measured after the patient has already exhibited myalgia or myopathy. It would be desirable to determine a subject's susceptibility to an HMG CoA reductase inhibitor induced myalgia or myopathy, and/or to predict the potential severity of HMG CoA reductase inhibitor induced myalgia or myopathy, prior to prescribing a particular HMG CoA reductase inhibitor to the subject. Such a test will greatly reduce the chance or likelihood of pain and suffering as well as the potential economic loss by the subject. Unfortunately, no such test is currently available.

Therefore, there is a need for a method of determining a subject's potential adverse reaction susceptibility to an HMG CoA reductase inhibitor.

### SUMMARY OF THE INVENTION

One aspect of the present invention is based on genotyping a CYP3A gene of a subject and utilizing that data for a variety of purposes. In one particular embodiment, the present invention provides a method for predicting the severity of adverse reaction in a subject about to receive an HMG CoA reductase inhibitor, a method of determining a suitable treatment for lowering a serum cholesterol level in a patient, and a method of determining an appropriate HMG CoA reductase inhibitor for a patient.

Some of the methods of the present invention comprise determining the cytochrome P450 3A (CYP3A) genotype of the subject. In some embodiments, CYP3A5 genotype of the subject is determined. Within these embodiments, the presence of CYP3A5*3 genotype, especially when the subject has homozygous CYP3A5*3 genotype, is used as an indication that the subject is likely to have an increased risk of susceptibility and/or severity for an adverse reaction to the HMG CoA reductase inhibitor. Knowing the subject's CYP3A5 genotype allows one to take an appropriate course of action or prescribe a proper treatment for the subject.

One particular aspect of the present invention provides a method for determining a susceptibility of an adverse reaction to an HMG CoA reductase inhibitor in a subject. The method generally comprises determining the CYP3A5 genotype of the subject. The presence of at least one CYP3A5*3 allele is an indication that the subject has increased likelihood of adverse reaction susceptibility or severity to the HMG CoA reductase inhibitor relative to those subjects that do not have any CYP3A5*3 allele.

In one particular embodiment, the method for determining the genotype comprises obtaining a genomic DNA sample from the subject; and analyzing the genomic DNA sample to determine the CYP3A5 genotype. In some embodiments, methods of the present invention comprise determining whether the subject has at least one CYP3A5*3 allele. Determination of CYP3A5 genotype can be achieved by analyzing a SNP that is in linkage disequilibrium with a particular CYP3A5 allele of interest, e.g., CYP3A5*3 allele, or by analyzing a haplotype that is associated with a particular CYP3A5 allele of interest, e.g., CYP3A5*3 allele.

The genomic DNA sample can be analyzed by using any of the methods known to one skilled in the art. In one particular embodiment, DNA sample analysis comprises amplifying at least a portion of the CYP3A5 gene using a primer pair to produce an amplified product, and analyzing the amplified product to determine the CYP3A5 genotype. There are various DNA analysis techniques available including Real-time PCR and Invader Assay.

In one particular embodiment, the DNA sample is analyzed by PCR using a primer pair. Within this embodiment, the primer pair can comprise 5′-CCT GCC TTC AAT TTT TCA CTG (forward) (SEQ ID NO: 1) and 5′-GCA ATG TAG GAA GGA GGG CT (reverse) (SEQ ID NO: 2).

Methods of the present invention are useful in determining a susceptibility and/or severity of an adverse reaction to an HMG CoA reductase inhibitor in which the HMG CoA reductase inhibitor is metabolized by an enzyme encoded by the CPY3A5 gene. Typically, such a HMG CoA reductase inhibitor is a statin drug. Exemplary statin drugs include atorvastatin, fluvastatin, lovastatin, simvastatin, pravastatin, rosuvastatin, and cerivastatin.

Another aspect of the present invention provides a method for determining a suitable treatment for lowering a serum cholesterol level in a patient. Such method comprises determining the CYP3A5 genotype of the patient, and when the patient has a homozygous CYP3A5*3 genotype, prescribing to the patient a serum cholesterol lowering drug in which the majority of the drug is metabolized by an enzyme other than the enzyme encoded by the CPY3A5 gene.

Yet another aspect of the present invention provides a method for determining cholesterol lowering treatment regimen in a patient. The method comprises determining the CYP3A5 genotype of the patient; and prescribing an HMG CoA reductase inhibitor to lower the patient's serum cholesterol when the patient's CYP3A5 genotype does not comprise CYP3A5*3 allele.

Still another aspect of the present invention provides a method for determining whether a patient is suitable for HMG CoA reductase inhibitor treatment to lower the patient's serum cholesterol level. The method comprises analyzing the CYP3A5 genotype of the patient; and identifying whether the patient has at least one CYP3A5*3 allele. When the patient has at least one CYP3A5*3 allele, it is an indication that the patient has increased risk of adverse reaction or severity to the HMG CoA reductase inhibitor relative to those without any CYP3A5*3 allele, and therefore may not be suitable for HMG CoA reductase inhibitor treatment.

In some embodiments, the step of analyzing the CYP3A5 genotype comprises analyzing a SNP that is in linkage disequilibrium with CYP3A5*3 allele. Within these embodiments, one can analyze the SNP that is located in the promoter region of the CYP3A4 gene to determine whether the patient has CYP3A5*3 allele. Alternatively, one can analyze the CYP3A5 genotype by analyzing a haplotype that is associated with CYP3A5*3 allele. Still alternatively, one can analyze nucleotide number 6986 of CYP3A5 gene to determine the presence of CYP3A5*3 allele.

### DETAILED DESCRIPTION OF THE INVENTION

Unless defined otherwise, all technical and scientific terms used herein have the meaning commonly understood by a person skilled in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

Various biochemical and molecular biology methods are well known in the art. For example, methods of isolation and purification of nucleic acids are described in detail in WO 97/10365, WO 97/27317, Chapter 3 of Laboratory Techniques in Biochemistry and Molecular Biology: Hybridization With Nucleic Acid Probes, Part I. Theory and Nucleic Acid Preparation, (P. Tijssen, ed.) Elsevier, N.Y. (1993); and Sambrook et al., Molecular Cloning: A Laboratory Manual, Cold Spring Harbor Press, N.Y., (1989); and Current Protocols in Molecular Biology, (Ausubel, F. M. et al., eds.) John Wiley & Sons, Inc., New York (1987-1999), including supplements such as supplement 46 (April 1999).

The terms “nucleic acid” “polynucleotide” and “oligonucleotide” are used interchangably herein and refer to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

A “gene,” for the purposes of the present disclosure, includes a DNA region encoding a gene product. The region can also include DNA regions that regulate the production of the gene product, whether or not such regulatory sequences are adjacent to coding and/or transcribed sequences. Accordingly, a gene can include, without limitation, promoter sequences, introns, terminators, translational regulatory sequences such as ribosome binding sites and internal ribosome entry sites, enhancers, silencers, insulators, boundary elements, replication origins, matrix attachment sites and locus control regions.

The term “detectably labeled” means that an agent (e.g., a probe) has been conjugated with a label that can be detected by physical, chemical, electromagnetic and other related analytical techniques. Examples of detectable labels that can be utilized include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

Overview

The present inventors have discovered that a subject's adverse reaction susceptibility to an HMG CoA reductase inhibitor is associated with the subject's certain genotype. As used in this invention, a “subject” or “patient” refers to a mammal, preferably human.

It is well recognized that different patients respond in different ways to the same drug or medication. These differences are often greater among members of a population than they are within the same person at different times (or between monozygotic twins). Vesell, “Pharmacogenetic perspectives gained from twin and family studies,” Pharmacol. Ther., 1989, 41, 535-552.

It is estimated that genetics can account for 20 to 95 percent of variability in drug disposition and effects. Kalow et al., “Hypothesis: comparisons of inter- and intra-individual variations can substitute for twin studies in drug research,” Pharmacogenetics, 1998, 8, 283-289. Although many nongenetic factors influence the effects of medications, including age, organ function, concomitant therapy, drug interactions, and the nature of the disease, there are now numerous examples of cases in which interindividual differences in drug response are due to sequence variants in genes encoding drug-metabolizing enzymes, drug transporters, or drug targets. Evans et al., “Pharmacogenomics: translating functional genomics into rational therapeutics,” Science, 1999, 286, 487-491; Evans et al., “Pharmacogenomics: the inherited basis for interindividual differences in drug response,” Annu Rev Genomics Hum Genet., 2001, 2, 9-39; and McLeod et al., “Pharmacogenomics: unlocking the human genome for better drug therapy,” Annu Rev Pharmacol Toxicol., 2001, 41, 101-121. Unlike other factors influencing drug response, inherited determinants generally remain stable throughout a person's lifetime.

The present inventors have found that a subject's adverse reaction susceptibility to HMG CoA reductase inhibitors that are metabolized by a certain cytochrome p450 enzyme can be partly characterized by genotyping. In particular, the present inventors have found that genotyping can measure a subject's adverse reaction susceptibility to statin drugs. As used herein, a “statin drug” refers to an HMG CoA reductase inhibitor, such as atorvastatin, cerivastatin, fluvastatin, lovastatin, pravastatin, rosuvastatin, and simvastatin. Preferably, methods of the present invention can be used to predict the susceptibility and/or severity of a potential adverse reaction, prior to initiating therapy with atorvastatin or any other statin that is metabolized by a certain cytochrome P450.

Cytochrome p450 enzymes (i.e., “P450s”) are often designated by the letters CYP followed by a set of letters and numbers that distinguish enzyme isoforms. P450s encompass a highly diverse “superfamily” of hemoproteins, and one of their most relevant functions is that of metabolizing drugs in humans. These enzymes are typically located in the endoplasmic reticulum and are highly concentrated in the liver and small intestine. Oxidative metabolism by cytochrome p450 enzymes is a primary method of drug metabolism. Drug metabolism can either activate or inactivate a drug. Drug metabolism can also alter the solubility of a drug or drug derivative, impacting its route of excretion from the body (renal, if water soluble; and hepatobiliary, if not water soluble).

Of the variety of P450s, it is believed that the cytochrome P450 3A (CPY3A) enzyme subfamily is the most abundant of the human cytochrome enzymes. CYP3A enzymes are a large group of monooxygenase enzymes responsible for the metabolism of potentially toxic organic molecules. NADPH is required as a coenzyme and O2 is used as a substrate.

Human CYP3A genes are localized in a cluster on chromosome 7 (7q21-q22.1). Finta et al., “The human cytochrome P450 3A locus. Gene evolution by capture of downstream exons,” Gene, 2000, 260, 13-23. It is believed that this locus contains four functional genes (CYP3A4, 3A43, 3A5, and 3A7) and two putative pseudogenes (CYP3AP1 and CYP3AP2). Id. While CYP3A7 is expressed to a greater degree during fetal life, CYP3A4 and CYP3A5 are believed to be the main isoforms expressed during adult life. Both are expressed by a variety of human tissues. Xie et al., “Genetic variability in CYP3A5 and its possible consequences,” Pharmacogenomics, 2004, 5, 243-272. Anttila et al., “Expression and localization of CYP3A4 and CYP3A5 in human lung,” Am J Respir Cell Mol Biol., 1997, 16, 242-249.

Studies have shown that there are several variant forms of CYP3A4 (Lamba et al., Pharmacogenetics, 2002, 12, 121-132) and CYP3A5 (Kuehl et al., Nat Genet., 2001, 27, 383-391). The most well characterized CYP3A4 polymorphism (i.e., allele) is CYP3A4*1B. Westlind et al., Biochem Biophys Res Commun., 1999, 259, 201-205, and Westlind, et al., Biochem Biophys Res Commun., 2001, 281, 1349-1355. This promoter variant may be associated with altered clearance of index substrates, e.g., drugs. Sata et al., Clin Pharmacol Ther., 2000, 67, 48-56; Hesselink et al., Clin Pharmacol Ther., 2003, 74, 245-254; and Hesselink et al., Clin Pharmacol Ther., 2004, 76, 545-556. However, this has not been a consistent observation. Floyd et al., Pharmacogenetics, 2003, 13, 595-606; Eap et al., Eur J Clin Pharmacol., 2004, 60, 231-236; and Lamba et al., Adv Drug Deliv Rev., 2002, 54, 1271-1294. In fact, recent data suggest that allelic association between CYP3A4*1B and wild type CYP3A5 may be the cause of these phenotypic changes. Lamba et al., Adv Drug Deliv Rev., 2002, 54, 1271-1294 and Dally et al., Cancer Lett., 2004, 207, 95-99.

Currently, wild type CYP3A5 (i.e., CYP3A5*1) is the only CYP3A5 allele known to encode a functional enzyme. Kuehl et al., Nat Genet., 2001, 27, 383-391. Further, wild type CYP3A5 is only expressed in a minority of the general population (from 10% in patients of European heritage to ˜40% in patients of African heritage). Lamba et al., Pharmacogenetics, 2002, 12, 121-132; and Kuehl et al., Nat Genet., 2001, 27, 383-391. The most common CYP3A5 polymorphism appears to be CYP3A5*3. Kuehl et al., Nat Genet, 2001, 27, 383-391. This allele contains a splice variant, which encodes a truncated non-functional protein. Lamba et al., Pharmacogenetics, 2002, 12, 121-132; Kuehl et al., Nat Genet., 2001, 27, 383-391.

The present inventors have found that testing a subject's CYP3A5 genotype allows one to predict the subject's severity and/or susceptibility of adverse reaction to a statin. In particular, the presence of CYP3A5*3 allele, especially homozygous CYP3A5*3 allele, is associated with the severity of statin-induced muscle damage. Methods of the present invention can be used clinically to prevent adverse drug reactions.

Currently, there are ten (10) known CYP3A5*3 alleles (from CYP3A5*3A to CYP3A5*3J). See http://www.imm.ki.se/CYPalleles/cyp3a5.htm. All CYP3A5*3 alleles contain the following single nucleotide polymorphism (i.e., SNP): 6986 A>G. Some of these CYP3A5*3 alleles contain other SNPs in addition to 6986 A>G. Regardless of the particular SNP combination, all CYP3A5*3 alleles result in the production of a substantially non-functional enzyme. Methods of the present invention involve detection of any of these ten CYP3A5*3 alleles.

It should be appreciated that analysis of CYP3A5 gene to determine whether a subject has CYP3A5*3 allele does not require a direct analysis of nucleotide sequence 6986 of CYP3A5 gene. For example, a promoter SNP in the CYP3A4 gene (CYP3A4*1B) is known to be in linkage disequilibrium (LD) with wild type CYP3A5. The term “linkage disequilibrium” refers to the co-occurrence of two alleles (e.g., SNPs or other nucleotide variations such as insertion, deletion, and microsatellites) at linked loci such that the frequency of the co-occurrence of the alleles is greater than would be expected from the separate frequencies of occurrence of each allele. Without being bound by any theory, it has been observed that subjects who inherit the CYP3A5*3 allele almost always inherit a normal wild type CYP3A4 gene. The present inventors have found that these alleles are highly associated (D′>0.87), i.e., in high linkage disequilibrium. Accordingly, analysis of CYP3A5 gene to determine whether the subject carries CYP3A5*3 allele can be achieved indirectly by analyzing CYP3A4 promoter SNP or any other SNP that is in linkage disequilibrium with CYP3A5*3 allele. As such, unless explicitly excluded, the terms “analysis of CYP3A5 gene,” “genotyping CYP3A5 gene,” and “determining CYP3A5 genotype” include indirect analysis/determination of CYP3A5 genotype. Indirect analysis of CYP3A5 genotype can be achieved by analyzing any nucleotide variant(s) (e.g., SNPs, insertion, deletion, and microsatellites) or haplotype(s) that are associated with a particular CYP3A5 genotype of interest, e.g., CYP3A5*3 allele. Haplotype refers to a particular set of genomic DNA variants (e.g., SNPs, insertion, deletion, and microsatellites) in a region of chromosome which are usually inherited as a unit. However, it should be appreciated that occasionally genetic rearrangements may occur within a haplotype block.

Detection Method

Some aspects of the present invention are based on genotyping a CYP3A5 gene of a subject and utilizing that information for a variety of purposes. In one particular embodiment, the present invention provides a method for predicting the severity of and/or susceptibility to adverse reaction in a subject about to receive an HMG CoA reductase inhibitor, a method of determining a suitable treatment for lowering a serum cholesterol level in a patient, and a method of determining an appropriate HMG CoA reductase inhibitor for a patient.

Some of the methods of the present invention comprise determining the CYP3A5 genotype of the subject. In some embodiments, the presence of CYP3A5*3 genotype, especially when the subject has homozygous CYP3A5*3 genotype, is used as an indication that the subject is likely to have an increased risk of susceptibility and/or severity for an adverse reaction to the HMG CoA reductase inhibitor.

Any genotyping method known in the art can be used to practice the methods of the present invention. Most conventional genotyping methods involve a primer defined amplification and the analysis of the amplified product. Such product can be analyzed by a variety of methods including, but are not limited to, one or more of the following techniques: restriction fragment length polymorphism (RFLP), electrophoresis, sequencing (including pyro sequencing), probe hybridization, disrupted probe hybridization, and mass spectrometer (including MALDI-TOF). Other suitable genotyping analytical methods include, but are not limited to, primer defined allele-, haplotype-, or sequence specific amplification.

In one particular embodiment, methods of the present invention comprise determining the subject's CYP3A5 genotype. Determination of the subject's CYP3A5 genotype generally involves obtaining a genomic DNA sample of the subject and analyzing the sample. While the scope of the present invention includes obtaining a sufficient amount of the subject's genomic DNA sample for a direct analysis, the detection method will now be described in reference to a method that includes amplifying the sample. Any one of a variety of nucleic acid amplification methods that are useful in increasing the number of copies of a polynucleotide of interest in the genomic DNA sample can be used. Such amplification methods are well known in the art, and they include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics, 1989, 4, 560; Landegren et al., Science, 1988, 241, 1077), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, and isothermal amplification methods such as nucleic acid sequence based amplification (NASBA), and self-sustained sequence replication (Guatelli et al., Proc. Natl. Acad. Sci. USA, 1990, 87, 1874). Based on such methodologies, and disclosures provided herein, a person skilled in the art can readily design primers in any suitable regions 5′ and 3′ to CYP3A gene of interested, particularly of CYP3A5 gene (GenBank Accession Nos. NM_{—}000777 and AC005020). Such primers may be used to amplify DNA of any length so long that it contains a sufficient number of CYP3A5 gene to allow detection of CYP3A5*3 allele. Since all currently known CYP3A5*3 genotypes contain 6986A>G mutation, it is preferred that the method includes amplifying base number 6986 of CYP3A5 gene. As stated above, the scope of the present invention includes indirectly genotyping CYP3A5 gene by analyzing any SNP or haplotype that is associated with a particular CYP3A5 genotype of interest.

As used herein, the terms “amplified polynucleotide” and “amplified product” are used interchangeably herein and refer to a primer defined nucleic acid molecule whose amount has been increased at least two fold by any nucleic acid amplification method performed in vitro as compared to its starting amount in a test sample. In other preferred embodiments, an amplified polynucleotide is the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified product is at least about 30 nucleotides in length. More typically, an amplified polynucleotide is at least about 50 nucleotides in length. In a preferred embodiment of the invention, an amplified polynucleotide is at least about 100 nucleotides in length. In yet another preferred embodiment of the invention, an amplified polynucleotide is at least about 200, 300, or 400 nucleotides in length. Irrespective of the length of an amplified product, it should be appreciated that the total length of an amplified product of the invention should be long enough to allow a sufficiently accurate genotyping of CYP3A5 gene.

In one embodiment, a fragment of genomic DNA sufficient to genotype CYP3A5 is amplified and the amplification product is detected by fluorescence resonance energy transfer (FRET) using labeled nucleic acids as internal hybridization probes. Other suitable hybridization probes include radiation labeled probes, luminescence or chemiluminescence detection, fluorescence detection, time-resolved fluorescence detection, fluorescence polarization, mass spectrometry, and electrical detection, as well as other types of probes known to one skilled in the art.

In FRET, a pair of labeling molecules that can undergo energy transfer when located close to each other (less than 6 nucleotides apart on a nucleotide sequence) to cause a change in emission intensity in at least one of the labeling molecules is used to make the labeled nucleic acids (i.e., probes). An example of a labeling molecule for one nucleic acid in a pair includes, but are not limited to, fluorescein. Examples of labeling molecules for the other nucleic acid in the pair include but are not limited to LC RED 640 (Roche Lightcycler), LC RED 705 (Roche Lightcycler).

Methods of the present invention can be practiced by employing a real-time PCR. In real-time PCR, internal hybridization probes are typically included in the PCR reaction mixture so that product detection occurs as the product is formed, reducing post-PCR processing time. Roche Lightcycler PCR instrument (U.S. Pat. No. 6,174,670) or other real-time PCR instruments can be used in this embodiment of the invention.

Another example of a suitable genotyping method is the Invader Assay, a commercially available kit that can be purchased from Third Wave Technologies, Inc. (TWT) in Madison, Wis., USA. See http://www.twt.com/invader_tech/inv_how.htm. In this method, cleavage enzymes (proprietary “cleavases”) are used to cleave hybrid DNA molecules formed between enzyme specific designer oligonucleotides (proprietary “Invader oligonucleotides”) and the target patient DNA. When the designer oligonucleotide is complementary to the patient DNA, cleavage occurs. When the patient DNA has a mismatch with the oligonucleotide, cleavage does not occur. Wild type and variant alleles are then differentially labeled with fluorescent dyes, and genotype is assigned using a fluorescence plate reader. An example of the application of this technology (TWT Invader assay) to CYP3A5 genotyping is illustrated in the Examples section below.

Additional objects, advantages, and novel features of this invention will become apparent to those skilled in the art upon examination of the following examples thereof, which are not intended to be limiting.

### EXAMPLES

Study subjects were enrolled at a large, horizontally-integrated, multispecialty group practice located in central Wisconsin. Patients were invited to participate if they had ever been treated with atorvastatin. Case assignment (i.e., status as a case versus control) was determined by criteria outlined below.

One hundred thirty-seven adult subjects (68 cases and 69 controls) agreed to participate in this study. Due to potentially confounding pharmacokinetic interactions, patients were excluded from the study if they had previously been diagnosed with kidney disease or liver disease. Patients were also excluded if they had pre-existing muscle disease. Patients were not excluded if they were taking concomitant medications.

Patients were eligible for consideration as control (i.e., no muscle damage) only if they had normal serum CK levels while taking atorvastatin. In order to meet criteria for case status, patients were required to have at least one documented laboratory test showing an elevated serum CK level while taking atorvastatin, and this test needed to have been obtained during a period when the patient was experiencing what they perceived to be muscle pain related to the use of the drug (CK levels were disqualified if they had been drawn during an evaluation for unstable angina or myocardial ischemia).

Informed consent was obtained and blood drawn by a certified phlebotomist. Genomic DNA was extracted using the Autopure LS large sample nucleic acid purification system (Gentra Systems Inc., Minneapolis, Minn., USA). DNA concentration was determined by optical density at 260 nM. Genomic DNA was used to identify the CYP3A4 and CYP3A5 polymorphisms within this study population, as outlined below.

Genotyping Platform

CYP3A4 and CYP3A5 genotype were determined using the Invader Assay (Third Wave Technologies, Inc., Madison, Wis., USA). Prior to application of the Invader Assay, genomic DNA was amplified by thermocycling using primers specified below.

For CYP3A4*1B, primers were 5′-TGG CTT GTT GGG ATG AAT TTC AAG (forward) (SEQ ID NO: 3) and 5′-TTA CTG GGG AGT CCA AGG GTT CTG (reverse) (SEQ ID NO: 4). Wandel et al., “CYP3A activity in African American and European American men: population differences and functional effect of the CYP3A4*1B5′-promoter region polymorphism,” Clin Pharmacol Ther, 2000, 68, 82-91. For CYP3A5*3 a nested approach was utilized generating sequential amplicons of 1442 and 462 bp, respectively. Initial CYP3A5*3 primers were 5′-CCT GCC TTC AAT TTT TCA CTG (forward) (SEQ ID NO: 1) and 5′-GCA ATG TAG GAA GGA GGG CT (reverse) (SEQ ID NO: 2). Nested CYP3A5*3 primers were 5′-TAA TAT TCT TTT TGA TAA TG (forward) (SEQ ID NO: 5) and 5′-CAT TCT TTC ACT AGC ACT GTT C (reverse) (SEQ ID NO: 6). Kuehl et al., Nat Genet, 2001, 27, 383-391. In order to validate the specificity of each amplification product, all sequences were checked for homology between CYP3A43 (GenBank Accession No. AF337813), CYP3A7 (GenBank Accession No. NM_{—}000765), CYP3A4 (GenBank Accession Nos. Ml 8907 and AF280107), and CYP3A5 (GenBank Accession Nos. NM_{—}000777 and AC005020).

Prior to study activation, the Invader Assay was tested using 66 anonymously donated DNA samples. Since the clinic sample population is >95% Caucasian, these samples are presumed to be from patients of European heritage. Observed allele frequencies were consistent with this assumption (data not shown). To further validate the assay, a panel of African-American genomic DNA samples was also purchased from Coriell Cell Repositories (Camden, NJ, USA) and used to confirm prior literature reports of ethnic variation in allele frequency. In both populations, CYP3A4*1B and CYP3A5*3 were distributed according to Hardy-Weinberg equilibrium and identified at frequencies consistent with existing literature (data not shown). Lamba et al., “Common allelic variants of cytochrome P4503A4 and their prevalence in different populations,” Pharmacogenetics, 2002, 12, 121-132; Kuehl et al., “Sequence diversity in CYP3A promoters and characterization of the genetic basis of polymorphic CYP3A5 expression,” Nat Genet, 2001, 27, 383-391; Ball et al., “Population distribution and effects on drug metabolism of a genetic variant in the 5′ promoter region of CYP3A4,” Clin Pharmacol Ther., 1999, 66, 288-294 and Garcia-Martin et al., “CYP3A4 variant alleles in white individuals with low CYP3A4 enzyme activity,” Clin Pharmacol Ther., 2002, 71, 196-204.

The Invader Assay was then used to determine the frequency of the CYP3A4*1B and CYP3A5*3 alleles in genomic DNA from 137 study subjects (68 case patients and 69 control patients). Pre-amplification of gene fragments containing the polymorphisms was performed prior to this assay. Cleavage enzymes were then used to selectively cleave a subset of hybrid DNA molecules formed between Invader oligonucleotides and the target patient DNA. For each individual polymorphism, an enzyme-specific designer (Invader) oligonucleotide was used to hybridize to the target DNA, generating a cleavable structure. When the oligonucleotide is complementary to the patient DNA, cleavage occurs. When the patient DNA has a mismatch with the oligonucleotide, cleavage does not occur. Wild type and variant alleles were assayed simultaneously. Each was differentially labeled with fluorescent dye. Samples were read on a Perkin Elmer CytoFlour 4000 fluorescence plate reader. Excitation occurred at 485 nM with reading at 530 nM for the first dye, and excitation occurred at 560 nM with reading at 620 nM for the second dye. Genotypes were determined by fluorescence ratio (allele 1 versus allele 2). If the ratio exceeded 5.0, samples were considered homozygous wild type (allele 1). If the ratio was 0.30 to 3.0, samples were considered heterozygous. When this ratio was <0.20, the samples were considered homozygous for allele 2. The genotyping results are shown in Tables 1 and 2.

Validation of Polymorphisms

To verify the accuracy of the Invader Assay for determination of CYP3A4 and CYP3A5 genotype, a subset of genomic DNA samples were also genotyped by sequencing. Amplicons were gel purified and sequenced using the Thermo Sequenase radiolabeled terminator cycle sequencing kit (USB Corp., Cleveland, Ohio, USA). Sequencing products were electrophoresed through a 6.5% polyacrylamide gel and visualized using the Molecular Dynamics STORM 860 phosphorimager (Amersham Pharmacia Biotech, Inc., Piscataway, N.J., USA).

Statistical Analysis

To assess the relationship between genotype and risk of atorvastatin induced muscle damage, allele frequency was calculated within groups (i.e., case versus control) and compared using Fisher's exact test. To assess the relationship between genotype and severity of atorvastatin induced muscle damage, the degree of muscle damage was determined in the case cohort only, by ranking subjects according to serum CK level. This continuous endpoint showed a skewed distribution in our study population. Comparisons between CYP3A genotype and the severity of atorvastatin-induced muscle damage were therefore conducted using non-parametric methods (Kruskal-Wallis test). Results were considered statistically significant when P≦0.05.

Results

Patient Summary

A total of 137 subjects were enrolled in this retrospective case-control study. Age and gender were both found to be unequally distributed (Wilcoxon Rank Sum Test: P=0.020 for age and P=0.001 for gender). Other covariates that differ between cases and controls included a high-density lipoprotein cholesterol level (P=0.021), the concomitant use of Niacin (P=0.033), a personal history of coronary artery disease (P=0.035), and a family history of coronary artery disease (P=0.005). Wilke et al., Pharmacogenet. Genomics, 2005, 15, 415-421.

CYP3A and Risk of Muscle Damage

To determine whether CYP3A4*1B or CYP3A5*3 were associated with the risk of atorvastatin induced muscle damage, genotype frequency was compared between cases and controls. Table 1 shows allele frequency for the two major CYP3A gene variants characterized in this example. The frequency of CYP3A4*1B and CYP3A5*3 were similar for cases and controls (Fishers exact test: P=0.519 for CYP3A4*1B and P=0.468 for CYP3A5*3).

TABLE 1
Distribution of CYP 3A genotypes

Genotype

| | Cases | Controls |
|---|---|---|
| Genotype - CYP3A | n (%) | n (%) |
| CYP3A4*1B | | |
| Homozygous wild type | 55 (90) | 62 (94) |
| Heterozygous | 5 (8) | 4 (6) |
| Homozygous variant | 1 (2) | 0 |
| CYP3A5*3 | | |
| Homozygous variant | 54 (82) | 56 (88) |
| Heterozygous | 12 (18) | 8 (12) |
| Homozygous wild type | 0 | 0 |

CYP3A and Severity of Muscle Damage

To determine whether CYP3A4*1B or CYP3A5*3 were associated with the severity of muscle damage, serum CK levels were ranked and compared between genotypes in the case cohort only. See Table 2. When the data were analyzed in the context of concomitant lipid-lowering medication, a statistically significant relationship was revealed between CYP3A5 genotype and the severity of atorvastatin-induced muscle damage. This association, between CYP3A5 genotype and degree of serum CK elevation, was strengthened as patients taking additional lipid-lowering medications were sequentially removed from the dataset (Table 2) (P=0.025 without gemfibrozil and P=0.010 without gemfibrozil and niacin).

TABLE 2
Impact of other lipid lowering medications on serum CK levels by genotype (cases only)

| | CYP3A4 genotype | | | | | | | | | CYP3A5 genotype | | | | | | | | |
|---|---|---|---|---|---|---|---|---|---|---|---|---|---|---|---|---|---|---|
| | Homozygous | | | | Heterozygous | | | | | Homozygous | | | | Heterozygous | | | | |
| | (CYP3A4*1A/*1A) | | | | (CYP3A4*1A/*1B) | | | | P- | (CYP3A5*3/*3) | | | | (CYP3A5*1/*3) | | | | P- |
| | n | Mean | SD | Median | n | Mean | SD | Median | value | n | Mean | SD | Median | n | Mean | SD | Median | value |
| No medications | 55 | 441.1 | 425.0 | 321.0 | 6 | 268.8 | 64.6 | 246.0 | 0.146 | 54 | 429.2 | 412.5 | 317.5 | 12 | 344.0 | 293.3 | 246.0 | 0.096 |
| removed | | | | | | | | | | | | | | | | | | |
| Less gemfibrozil | 50 | 434.6 | 428.9 | 316.5 | 6 | 268.8 | 64.6 | 246.0 | 0.153 | 49 | 441.9 | 430.5 | 321.0 | 11 | 261.7 | 72.7 | 239.0 | 0.025^{a} |
| Less gemfibrozil | 46 | 420.4 | 411.2 | 316.5 | 5 | 246.6 | 38.9 | 239.0 | 0.059 | 45 | 428.0 | 412.9 | 321.0 | 10 | 249.9 | 64.5 | 237.5 | 0.010^{a} |
| AND less niacin | | | | | | | | | | | | | | | | | | |

^{a}Kruskal-Wallis test.
CK, creatine kinase.

Discussion

It is believed that atorvastatin acid is hydroxylated by members of the CYP3A enzyme family. A study was conducted to determine whether patients expressing CYP3A gene variants were at increased risk for the development of an atorvastatin induced adverse drug reaction. Using a retrospective case-control study design, 137 study subjects (68 cases and 69 controls) were genotyped for the two most common functionally relevant CYP3A gene polymorphisms, CYP3A4*1B and CYP3A5*3.

Data analysis revealed an association between CYP3A genotype and serum CK levels in case patients, and this interaction was strengthened in the absence of other lipid lowering medications. In a tiered analysis conducted on case subjects, it was found that the CYP3A5*3/*3 (homozygous) genotype was associated with a greater degree of serum CK elevation than the CYP3A5*1/*3 (heterozygous) genotype, particularly after subjects using other lipid lowering agents were removed from the dataset (Table 2).

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All publications disclosed herein are incorporated by reference in their entirety.

What is claimed is:

1. A method for determining a susceptibility of an adverse reaction to an HMG CoA reductase inhibitor in a subject, said method comprising determining the CYP3A5 genotype of the subject, wherein the presence of at least one CYP3A5*3 allele is an indication of the subject's increased adverse reaction susceptibility or severity to the HMG CoA reductase inhibitor relative to those without at least one CYP3A5*3 allele.

2. The method of claim 1, wherein said method of determining the genotype comprises:
obtaining a genomic DNA sample from the subject; and
analyzing the genomic DNA sample to determine the CYP3A5 genotype.

3. The method of claim 2, wherein said step of analyzing the genomic DNA sample comprises analyzing a SNP that is in linkage disequilibrium with CYP3A5*3 allele.

4. The method of claim 2, wherein said step of analyzing the genomic DNA sample comprises analyzing a haplotype that is associated with CYP3A5*3 allele.

5. The method of claim 2, wherein said method of analyzing the genomic DNA sample comprises:
amplifying at least a portion of the CYP3A5 gene using a primer pair to produce an amplified product; and
analyzing the amplified product to determine the CYP3A5 genotype.

6. The method of claim 2, wherein said method of analyzing the genomic DNA sample comprises Real-time PCR or Invader Assay.

7. The method of claim 5, wherein one of the primer pair has a length of from about 12 to 50 nucleotide residues and is either homologous with or complementary to at least 12 consecutive nucleotides SEQ ID NO: 1 and the other primer pair has a length of from about 12 to 50 nucleotide residues and is either homologous with or complementary to at least 12 consecutive nucleotides of SEQ ID NO: 2.

8. The method of claim 7, wherein the primer pair comprises SEQ ID NO: 1 and SEQ ID NO: 2 or complementary pair thereof.

9. The method of claim 1, wherein the HMG CoA reductase inhibitor is metabolized by an enzyme encoded by CPY3A5 gene.

10. The method of claim 1, wherein the HMG CoA reductase inhibitor is a statin drug.

11. The method of claim 10, wherein the statin drug is selected from the group consisting of: atorvastatin, fluvastatin, lovastatin, simvastatin, pravastatin, rosuvastatin, and cerivastatin.

12. A method of determining a suitable treatment for lowering a serum cholesterol level in a patient, said method comprising:
determining the CYP3A5 genotype of the patient,
wherein when the patient has a homozygous CYP3A5*3 genotype, prescribing to the patient a serum cholesterol lowering drug in which the majority of the drug is metabolized by an enzyme other than the enzyme encoded by the CPY3A5 gene.

13. The method of claim 12, wherein the serum cholesterol lowering drug is an HMG CoA reductase inhibitor.

14. The method of claim 13, wherein the HMG CoA reductase inhibitor is a statin drug.

15. The method of claim 12, wherein the serum cholesterol lowering drug is not an HMG CoA reductase inhibitor.

16. A method for determining cholesterol lowering treatment regimen in a patient, said method comprising:
determining the CYP3A5 genotype of the patient; and
prescribing an HMG CoA reductase inhibitor to lower the patient's serum cholesterol when the patient's CYP3A5 genotype does not comprise CYP3A5*3 allele.

17. A method for determining whether a patient is suitable for HMG CoA reductase inhibitor treatment to lower the patient's serum cholesterol level, said method comprising:
analyzing the CYP3A5 genotype of the patient; and
identifying whether the patient has at least one CYP3A5*3 allele,
wherein the presence of at least one CYP3A5*3 allele is an indication that the patient has increased risk of adverse reaction or severity to the HMG CoA reductase inhibitor relative to those without any CYP3A5*3 allele, and therefore may not be suitable for HMG CoA reductase inhibitor treatment.

18. The method of claim 17, wherein said step of analyzing the CYP3A5 genotype comprises analyzing a SNP that is in linkage disequilibrium with CYP3A5*3 allele.

19. The method of claim 18, wherein the SNP is located in the promoter region of the CYP3A4 gene.

20. The method of claim 17, wherein said step of analyzing the CYP3A5 genotype comprises analyzing a haplotype that is associated with CYP3A5*3 allele.

21. The method of claim 17, wherein said step of analyzing the CYP3A5 genotype comprises analyzing nucleotide number 6986 of CYP3A5 gene.
